(19) United States
(12) Patent Application Publication
(10) Pub. No.: US 2007/0197748 A1
(43) Pub. Date: Aug. 23, 2007

(54) Mixture of dodecanethiols, method for the production and use thereof

(76) Inventors: Rosangela Pirri, Montardon (FR); George Fremy, Sauveterre de Bearn (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 10/588,422

(22) PCT Filed: Feb. 4, 2005
(86) PCT No.: PCT/FR05/00260

§ 371 Date: Aug. 4, 2006

(30) Foreign Application Priority Data
Feb. 6, 2004 (FR) 0401154

(51) Int. Cl. C08F 4/00; C07C 319/02; C07C 319/16

(52) U.S. Cl. 526/223; 568/21; 568/70

(57) ABSTRACT

Process for the preparation of tert-dodecyl mercaptan by reaction of hydrogen sulfide with tri(n-butene) in the presence of a catalyst. tert-Dodecyl mercaptan thus prepared and its use as chain-transfer agent in radical (co)polymerization reactions.

The present invention relates to a mixture of dodecane-thiols, to its process of manufacture and to its uses, in particular as chain-transfer agent in radical poly-merization reactions.

Application FR 2 531 426 teaches a process for the preparation of tert-alkyl mercaptans, in particular of tert-dodecyl mercaptans, by the action of hydrogen sulfide on an olefin. This document specifically discloses carrying out the process employed on the industrial scale and using tetrapropylene as starting material in order to synthesize the tert-dodecyl mercaptan hereinafter referred to as TDM 4P. This document also mentions the use of this type of process starting from triisobutylene, which results in a mixture of 2 isomers of dodecene: 4,4-dimethyl-2-neo-pentyl-1-pentene and 2,2,4,6,6-pentamethyl-3-heptene, giving a mixture of the two corresponding isomers of tert-dodecyl mercaptan, hereinafter referred to as TDM 3B.

Alkyl mercaptans, in particular tert-alkyl mercaptans, are widely used in industrial polymerization reactions as they make it possible to reduce the length of the polymer chains and to lower their molecular weight. The polymers thus obtained have a lower viscosity, which is often necessary in certain applications, for example for easy forming of the plastic by standard techniques, such as injection molding.

Tetrapropylene, which is available commercially, is obtained by oligomerization of propene under controlled temperature and pressure conditions in the presence either of an acid catalyst, such as inorganic acids, for example H₃PO₄ and H₂SO₄, or of an organometallic catalyst, such as alkylaluminums or alkali metals. Tetrapropylene corresponds to a mixture of alkenes having from 10 to 14 carbon atoms (C₁₀ to C₁₄ mono-olefins) in which the content of alkene comprising 12 carbon atoms (also referred to as dodene or C₁₂ olefin) is between 55 and 85% and more specifically between 60 and 80% by weight. Each of these alkenes is present in said mixture in the form of positional isomers of the double bond and of geometric isomers. Dodecene corres-ponds to the tetramer of propene and the presence (in the product from the reaction for the oligomerization of propene resulting in C₁₂) of C₁₀, C₁₁, C₁₃ and C₁₄ olefins results from side reactions.

It is always desirable to have available a broadened range of TDM in order to be able to better respond to the requirements of the market, both technically, depending on the applications targeted, and economically.

A subject matter of the present invention is thus a mixture of isomers of dodecanethiol prepared by catalytic reaction of hydrogen sulfide with the trimer of n-butene and exhibiting a diagram of distillation temperatures, at 19 millibar, such that point 50 is 123° C.±1° C. and that the difference in temperature between point 20 and point 80 is less than or equal to 4° C.

Point 20, 50 and 80 of the distillation is understood to mean the temperature at which respectively 20, 50 and 80% by weight of the mixture has passed into the vapor phase.

Another subject matter of the present invention is the process for the preparation of the mixture of isomers of dodecanethiol, characterized in that it consists in reacting hydrogen sulfide with tri(n-butene) in the presence of a catalyst.

The term “tri(n-butene)” is intended to denote a mixture of monoolefins obtained by oligomerization of n-butene in the presence of a catalytic system which is often identical to that mentioned above. The alkenes present in said mixture comprise generally from 11 to 13 carbon atoms with a content by weight of dodecene of greater than 90% and even of greater than 95% by weight. Tri(n-butene) is available commercially.

Surprisingly, it has been found that the process according to the invention leads to a different product from those resulting disclosed in patent FR 2 531 426. Specifically, the product, which is a mixture of isomers, exhibits a diagram of distillation tempera-tures which are substantially different from those of the TDM mixtures obtained from tetrapropylene or triisobutene.

This difference is explained by the nature of the tri(n-butene) reactant used, which results in a homo-geneity in behavior of the isomers of the mixture which is much greater than those of the TDM 4P products. As regards the comparison with the TDM 3B products, it may be noted that the mixtures of the invention have a point 50 which is substantially greater than the boiling point of the TDM which behaves like a virtually pure substance. In addition to this difference, it should be emphasized that the synthesis of the mixtures of the invention, which is very close as regards the process level to the synthesis employed with the TDM 4P products, avoids the problems of thermal stability of the intermediates which the synthesis of the TDM 3B products exhibits.

The distillation is carried out on a Cadiot “spinning band” column without reflux and at a pressure of 19 mbar. Measurements carried out on the TDM 4P and the mixture of the invention are given in table 1 below.

TABLE 1

| | Distillation | % Mixture of | Distillation |
|---|---|---|---|
| % TDM | temperatures | the invention | temperatures |
| distilled | (° C.) | distilled | (° C.) |
| 10% | 97 | 10% | 116 |
| 20% | 106 | 19% | 122 |
| 29% | 109 | 29% | 122 |
| 40% | 113 | 40% | 123 |
| 54% | 114 | 49% | 123 |
| 64% | 118 | 59% | 123 |
| 72% | 118 | 70% | 124 |
| 81% | 121 | 83% | 125 |

The mixture of the invention is, industrially, a complex mixture comprising thiols ranging from 11 to 13 carbon atoms but with a content of dodecanethiol (12 carbon atoms) of greater than 90%, preferably of greater than 95%, by weight.

By way of comparison, the mixture obtained from tetrapropylene has a distillation diagram which is substantially different in terms of level, point 50 being 10° C. lower; the difference between points 20 and 50 is 15° C. for the TDM 4P, to be compared with the 3 or 4° C. of the mixtures of the invention.

The catalyst which can be used in the process according to the invention can be chosen from an acid compound, a metal oxide or a combination of these 2 products. Use may be made, as acid compound, of a Lewis and/or Bronsted acid which is solid or liquid and which is miscible or immiscible in the reaction medium, for example chosen from an organic or inorganic acid, an alumina, a clay, a silica or a silica-alumina, a zeolite, a heteropolyacid or a weakly or strongly acidic cation-exchange resin. Mention may be made, among the metals having an oxide which can be used as catalyst, of chromium, cobalt, molybdenum, tungsten, zirconium, niobium or nickel.

It is preferable to use a cation-exchange resin as catalyst.

The various polymers and copolymers comprising acid functional groups known to a person skilled in the art as cation exchangers are thus suitable. In particular, use may be made of resins based on sulfonated polystyrenes which are crosslinked, in particular with divinylbenzene, acrylic or phenylacrylic resins comprising free carboxyl groups, resins of phenol-formaldehyde type derived from phenolsulfonic acids, or lignosulfonic exchangers. Resins of this type are sold under various names.

More preferably still, use is made of a sulfonated styrene/divinylbenzene copolymer resin available commercially under the name Amberlyst® 15.

The molar ratio of the hydrogen sulfide to the olefin is at least 1 (corresponding to stoichiometry) and is generally between 1 and 100, preferably between 1 and 20, more preferably still between 1 and 5.

The temperature at which the process according to the invention is carried out depends on the catalyst used and is generally between 10 and 250° C., preferably between 50 and 150° C. and more preferably still between 70 and 120° C. The pressure can be equal to or greater than atmospheric pressure. It is generally between 5 and 80 bar, preferably between 10 and 50 bar and more preferably still between 10 and 20 bar.

The process can be carried out continuously or batch-wise. Preferably, it is carried out continuously.

The hourly space velocity, defined as being the ratio of the hourly flow rate by volume of olefin to the volume of catalyst, depends strongly on the activity of the catalyst used. It is generally between 0.01 and 100 h⁻¹, preferably between 0.1 and 10 h⁻¹ and more preferably still between 0.2 and 2 h⁻¹.

The reaction products, which can comprise the mercaptans formed, the unreacted hydrogen sulfide and the unconverted olefins, are separated using all the conventional methods known to a person skilled in the art. Use is generally made of distillation under reduced pressure to separate the unconverted olefins from the mercaptans formed.

Another subject matter of the present invention is the uses as chain-transfer agent in radical (co)polymer-ization reactions or as starting material in the synthesis of polysulfides.

When the mixture of the invention is used as chain-transfer agent in radical polymerization reactions, it makes it possible to obtain polymers (or plastics) having a substantially reduced viscosity. In addition, when it is used in the manufacture of polysulfides, it also results in a final material exhibiting substantial differences with regard to viscosity. Such properties are particularly advantageous at a practical level. For example, they make possible faster forming of the plastic by standard injection molding techniques, thus allowing an increase in the productive output of lines for the production of molded objects. They also make it possible, for example in the case of a styrene/butadiene copolymer, to reduce the amount of transfer agent which is necessary for the manufacture of a copolymer having the required Theological property, for example for the purpose of its application as binding agent for the coating of paper.

The present invention is illustrated by the following examples, which are given purely by way of illustration of the invention and should not be used to limit the scope thereof.

### EXAMPLE 1

Preparation of the Mixture from Tri(n-butene):

Use is made, as tri(n-butene), of a commercial mixture of alkenes comprising from 11 to 13 carbon atoms, the dodecene content of which is 98% by weight. The composition of the mixture is determined by gas chromatography coupled to mass spectrometry with (for the latter) a technique for detection by positive chemical ionization with ammonia.

200 ml of dry sulfonated styrene/divinylbenzene copoly-mer resin (known under the name Amberlyst® 15) are placed in a tubular reactor with a length of 680 mm and an internal diameter of 27 mm. Liquid tri(n-butene) is introduced continuously into this reactor, under a pressure of 15 bar, at the rate of 79 ml/h (i.e., 60.8 g/h or alternatively 363 mmol/h). At the same time, 26 normal liters/h (i.e., 36.9 g/h or alternatively 1083 mmol/h) of gaseous H₂S are injected therein. The H₂S/olefin molar ratio is 3.

The reaction medium is maintained at the temperature of 90° C.±2° C. The reactants are intimately mixed before they pass into the reactor. The liquid flowing out continuously from the reactor is collected and the remaining hydrogen sulfide is degassed.

This liquid is analyzed by gas chromatography coupled to mass spectrometry. The chromatograph used is equipped with a flame ionization detector and with a polar column. A chromatogram is observed comprising 2 clumps of peaks, corresponding to a retention time of between 2.7 and 4.2 minutes, on the one hand, and 4.3 and 9.6 minutes, on the other hand. These clumps are identified as corresponding respectively to the tri(n-butene) and to the mixture. The measurement of the areas corresponding to these 2 clumps makes it possible, after calibration, to calculate a percentage by weight of unconverted tri(n-butene) of 28% and a degree of conversion of 68%. Furthermore, the absence of other compounds shows that the degree of selectivity for the mixture is 100%.

### EXAMPLE 2

Use of the Mixture Prepared in Example 1 as Chain-Transfer Agent in the Radical Co-Polymerization of Styrene and Butadiene:

The following are introduced into a 1 liter reactor containing 450 g of water:
0.072 g of FeSO₄ and 0.19 g of sodium salt of hydroxymethanesulfinic acid (of formula: HO-CH₂-SO₂Na), corresponding to 2 of the 3 components of the initiating system
0.08 g of EDTA (ethylenediaminetetraacetate)
0.5 g of sodium phosphate
43.3 g of a 25% by weight aqueous potassium rosinate mixture (emulsifier)
76.94 g of styrene
163.59 g of butadiene
0.43 g of the chain-transfer agent prepared in example 1.

The above ingredients are added to the water at ambient temperature with stirring, so as to obtain an emulsion.

This emulsion is cooled to 9.5° C. and 0.35 g of para-menthane hydroperoxide (incorporated in a proportion of 55% by weight in water), which corresponds to the 3rd component of the initiating system, is added.

The polymerization begins from the introduction of the para-menthane hydroperoxide and stirring of the reaction medium at 150 revolutions/min is continued for 5 hours.

The polymerization is halted by adding, to the medium, the mixture comprising 0.077 g of potassium hydroxide, 0.072 g of sodium dithiocarbamate and 0.085 g of diethylhydroxylamine, made up to 15 g with water.

The latex obtained has a solids content of approximately 19% by weight.

After removing the residual monomers, a film of styrene/butadiene copolymer with a thickness of approximately 1 mm is obtained by drying the latex.

The melt viscosity of the film is measured at 100° C. and for a frequency of 1 Hz using a controlled-stress rheometer.

A value for this viscosity of 1.1×10⁵ Pa·s is obtained.

### EXAMPLE 3

(Comparative): Preparation of TDM 4P from Tetrapropylene:

Use is made, as tetrapropylene, of a commercial mixture of alkenes comprising from 10 to 14 carbon atoms, the dodecene content of which is 60%. The composition of this mixture is determined by gas chromatography coupled to mass spectrometry with (for the latter) a technique for detection by positive chemical ionization with ammonia.

Example 1 is repeated, the tri(n-butene) being replaced with the tetrapropylene.

A percentage by weight of unconverted tetrapropylene of 25% and a degree of conversion of 71% are measured. Furthermore, the absence of other compounds shows that the degree of selectivity for TDM is 100%.

### EXAMPLE 4

(Comparative): Use of the TDM 4P Prepared in Example 3 as Chain-Transfer Agent in the Radical Copolymerization of Styrene and Butadiene:

Example 2 is repeated, the tert-dodecyl mercaptan prepared in example 1 being replaced with the product obtained in example 3.

A value of 1.4×10⁵ Pa·s is obtained for the viscosity.

### EXAMPLE 5

Preparation of Di(tert-dodecyl) Trisulfide by Reaction of TDM 4P with Sulfur in the Presence of a Basic Catalyst

The synthesis of di(tert-dodecyl) trisulfide by reaction of TDM 4P with sulfur is carried out in the presence of a TPS catalyst (polyethoxylated tert-dodecyl mercaptan comprising 2.5% of sodium hydroxide).

The equipment is a jacketed 250 ml glass reactor equipped with a sintered glass filter situated in front of the discharge valve, with an inlet for the nitrogen via a dip pipe terminated by a sintered glass sparger, the nitrogen flow rate being controlled by a ball flow meter, with a water-cooled reflux condenser connected to the suction of the hood via an oil-comprising bubble counter, with a thermostatically controlled bath which makes possible the circulation of oil in the jacket, with a glass stirrer terminated by a PTFE anchor and equipped with a stirrer motor possessing a speed counter, and with a thermometer probe in a glass tube.

The operating conditions are as follows:

The reactor is placed beforehand under a nitrogen atmosphere and 2 g of TPS catalyst (cata/RSH=1%) are introduced with 202 g of TDM 4P (1 mol). The reactor is then heated to 120° C. Subsequently, 32 g of sulfur (1 mol) are introduced portionwise. Stirring is maintained at 500 rev/min. The complete dissolution of the sulfur takes place in approximately 45 minutes. Bubbling of nitrogen (flow rate =34 l/h) is then applied for the duration of the reaction.

Samples are taken over time (t=0 when the sulfur is introduced) in order to determine the residual mercaptan content (% by weight) by argentometry and the free sulfur content by HPLC. The percentage by weight of residual mercaptan makes it possible to access the conversion of the mercaptan. The conversion of TDM 4P is 92% after reacting for 2.5 hours.

The reaction mixture is subsequently cooled to 60° C., at which temperature 300 mmol of ethylene oxide (13.2 g) are introduced and allowed to react with the residual TDM for 3 hours. The reaction medium is subsequently stripped under nitrogen. The residual mercaptan content is less than 150 ppm after reaction.

At the end of the reaction, the polysulfide is filtered under hot conditions (40° C.) on the sintered glass filter of the reactor.

A sample of the product obtained is referred to as TDM 4P S3.

### EXAMPLE 6

Synthesis of Di(tert-dodecyl) Trisulfide by Reaction of the Mixture from Example 1 (hereinafter referred to as TERDAN) with Sulfur in the Presence of TPS Catalyst (polyethoxylated tert-dodecyl mercaptan comprising 2.5% of sodium hydroxide)

Use is made of the same procedure as in example 5 and a conversion of the TERDAN is obtained which is equivalent to that obtained with the TDM 4P during example 5 (92%).

After treatment with ethylene oxide and filtration, a sample is isolated which is referred to as TERDAN S3.

### EXAMPLE 7

Preparation of Di(tert-dodecyl) Pentasulfide by Reaction of TDM 4P with Sulfur in the Presence of a Basic TPS Catalyst

Use is made of the same equipment as for example 5 and the reaction is carried out as follows. The reactor is placed beforehand under a nitrogen atmosphere and then 202 g of TDM 4P (1 mol) are introduced in the presence of 1 g of TPS catalyst (cata/RSH=0.5%). The reactor is then heated to 90° C. and then 64 g of sulfur (2 mol) are subsequently introduced portionwise. Stirring is maintained at 500 rev/min. The complete dissolution of the sulfur takes place in approximately 60 minutes. Bubbling of nitrogen (flow rate =6 l/h) is then applied throughout the duration of the reaction.

The withdrawing of samples is carried out as in example 5. The conversion of the TDM 4P is greater than 99.5% after 4 hours.

The reaction mixture is subsequently cooled to 60° C., at which temperature 10 mmol of ethylene oxide (0.44 g) are introduced and are left to react with the residual TDM for 1 h. The reaction medium is subsequently stripped under nitrogen. The residual mercaptan content is less than 150 ppm after reaction.

At the end of the reaction, the polysulfide is filtered under hot conditions (40° C.) on the sintered glass filter of the reactor.

A sample of the product is withdrawn and referred to as TDM 4P S5.

### EXAMPLE 8

Synthesis of Di(tert-dodecyl) Pentasulfide by Reaction of the TERDAN with Sulfur in the Presence of TPS Catalyst

Example 7 is repeated, the TDM 4P being replaced with the TERDAN, and comparable results are obtained with regard to the conversion of the TERDAN (>99.5% after 4 h).

A sample is withdrawn and referred to as TERDAN S5.

The kinematic viscosities of the polysulfides are then measured according to standard ASTM D445 and are given in table 2.

TABLE 2

| | Viscosity at 40° C. (mm²/s) |
|---|---|
| Example 5: TDM S3 | 65 |
| Example 6: TERDAN S3 | 43 |
| Example 7: TDM S5 | 121 |
| Example 8: TERDAN S5 | 83 |

These viscosities show that polysulfides of greater fluidity are obtained when the TERDAN is used instead of the TDM 4P.

1. A mixture of isomers of dodecanethiol prepared by a process of reacting hydrogen sulfide with a trimer of n-butene in the presence of a catalyst, said mixture of isomers exhibiting a diagram of distillation temperatures, at 19 millibar, such that point 50 is 123° C.±1° C. and that the difference in temperature between point 20 and point 80 is less than or equal to 4° C.

2. The mixture as claimed in claim 1, characterized in that the catalyst is chosen from an acid compound, a metal oxide or a combination thereof.

3. The mixture as claimed in claim 1, characterized in that the catalyst is a cation-exchange resin.

4. The mixture as claimed in claim 1, characterized in that the catalyst is a copolymer of sulfonated styrene with divinylbenzene.

5. The mixture as claimed in claim 1, characterized in that the molar ratio of the hydrogen sulfide to the trimer of n-butene is between 1 and 100.

6. The mixture as claimed in claim 1, characterized in that the molar ratio of the hydrogen sulfide to the trimer of n-butene is between 1 and 5.

7. The mixture as claimed in claim 1, characterized in that the process is carried out at a temperature of between 10 and 250° C. and at a pressure of between 5 and 80 bar.

8. The mixture as claimed in claim 1, characterized in that the process is carried out at a temperature of between 50 and 150° C. and at a pressure of between 10 and 50 bar.

9. The mixture as claimed in claim 1, characterized in that the process is carried out at a temperature of between 70 and 120° C. and at a pressure of between 10 and 20 bar.

10. A process for the preparation of the mixture of claim 1, characterized in that it comprises the reaction of hydrogen sulfide with tri(n-butene) in the presence of an acid catalyst.

11. A process for radical (co)polymerization, characterized in that it is carried out in the presence of the mixture as claimed in claim 1 used as chain-transfer agent.

12. A process for the synthesis of di(tert-dodecyl) polysulfides, characterized in that it is carried out by reaction of the mixture as claimed in claim 1 with sulfur in the presence of a basic catalyst.

13. The mixture as claimed in claim 1, characterized in that the molar ratio of the hydrogen sulfide to the trimer of n-butene is between 1 and 20.

14. A process for preparing a mixture of isomers of dodecanethiol comprising reacting hydrogen sulfide with an olefin in the presence of a catalyst, said mixture of isomers exhibiting a diagram of distillation temperatures, at 19 millibar, such that point 50 is 123° C.±1° C. and that the difference in temperature between point 20 and point 80 is less than or equal to 4° C.

15. The process of claim 14, characterized in that said olefin is a trimer of n-butene.

16. The process as claimed in claim 14, characterized in that the catalyst is chosen from an acid compound, a metal oxide or a combination thereof.

17. The process as claimed in claim 14, characterized in that the catalyst is a cation-exchange resin.

18. The process as claimed in claim 14, characterized in that the catalyst is a copolymer of sulfonated styrene with divinylbenzene.

19. The process as claimed in claim 14, characterized in that the molar ratio of the hydrogen sulfide to the olefin is between 1 and 100.

20. The process as claimed in claim 14, characterized in that the process is carried out at a temperature of between 10 and 250° C. and at a pressure of between 5 and 80 bar.
